(19) United States
(12) Patent Application Publication
(10) Pub. No.: US 2009/0215753 A1
(43) Pub. Date: Aug. 27, 2009

(54) Topical Compositions for Paediatric Use

(75) Inventors: John Staniforth, Wiltshire (GB); Paul Goggin, Wiltshire (GB)

Correspondence Address: Davidson, Davidson & Kappel, LLC, 485 7th Avenue, 14th Floor, New York, NY, 10018, US

(73) Assignee: Pharmacodex Ltd., Chippenham, Wiltshire (GB)

(21) Appl. No.: 12/086,043

(22) PCT Filed: Dec. 7, 2006
(86) PCT No.: PCT/GB2006/050433

§ 371 Date: Apr. 21, 2009

(30) Foreign Application Priority Data
Dec. 7, 2005 (GB) 0524959.4
Dec. 7, 2005 (GB) 0524961.0

(51) Int. Cl. A61K 31/5513; A61P 25/08; A61K 31/4178; A61K 31/435; A61M 35/00

(52) U.S. Cl. 514/221; 514/397; 514/338; 604/289

(57) ABSTRACT

The present invention provides pharmaceutical compositions for treating a paediatric mammal, the compositions comprising a therapeutic agent and a pharmaceutically acceptable carrier, the composition being suitable for topical application resulting in transdermal administration of the therapeutic agent and a systemic therapeutic effect. The invention also provides relates uses of the compositions, applicators and kits.

The present invention relates to compositions suitable for paediatric use, for treating or preventing various disorders by transdermal administration of one or more therapeutic agents.

Administration of drugs to children, and to young children, infants and neonates in particular, presents a problem. Children can be non-compliant, and easily become upset by having to take medication.

In addition, many drugs used for the diseases of childhood are only licensed for use in older children or adults. Indeed, it has been estimated that only about 20 percent of drugs approved by the FDA have, until recently, been labeled for pediatric use. In the absence of clear dosing guidance for infants/young children, physicians have been forced to estimate doses from adult values based on the child's size or surface area. In doing so, children have been considered as “small adults”.

However, recent studies have demonstrated that paediatric metabolic rate, and consequently metabolism of drugs is affected by many factors, including the dynamics of growth and maturation of organs, changes in metabolism throughout infancy and childhood, changes in body proportion, and other developmental changes. It was found that children have a comparable, and sometimes higher metabolic clearance rate than adults for some drugs. For example, the greater tolerance of children to many anticancer drugs is thought to be attributable to their higher rates of metabolic clearance. This can mean that larger doses are required, and this can lead to difficulties in administration using conventional routes.

Conventionally, the most common route of administering pharmaceutically active agents is the oral route. However, this route of administration has drawbacks. Oral dosage forms, such as tablets, can be difficult for younger patients to swallow and there is a real risk of choking. Also, active agents presented for oral administration to the gastrointestinal tract also tend to have a relatively slow onset of action and so such presentations are not recommended for rescue situations, where intervention is required quickly in order to alleviate or avoid symptoms, for example, where rapid relief from bronchoconstriction is required during an asthma attack, or from fitting during an epileptic episode. Furthermore, in situations were the patient is nauseous or vomiting, for example, when undergoing chemotherapy, the patient neither wants to take a medicine orally, nor is administration via this route likely to be effective.

Thus, alternative routes of administration must be considered. Injections and suppositories are invasive and can be unpleasant, particularly for the young. In addition, not all drugs can be administered by these routes. Some active agents can be administered by inhalation, for example using nebulizers, pressurized metered dose inhalers (pMDIs), or dry powder inhalers (DPIs). However, these methods of administration have drawbacks, particularly for children.

pMDIs require co-ordination and good timing between activation of the inhaler and inhalation in order to ensure that a reasonable amount of the dose dispersed is actually inhaled and reaches the deep or lower lung. The young can have difficulty with this co-ordination. Some DPIs are breath-actuates in order to avoid the need for such co-ordination but, they too have drawbacks. The patient is frequently required to take a deep breath in order to activate the device. Also, it is often important that exhalation into the mouthpiece prior to inhalation does not occur as moisture from the breath can prevent the inhaler from functioning correctly. Thus, the use of a DPI can also be difficult, especially for children.

As a result of these difficulties associated with children using pMDIs and DPIs, these devices are not recommended for neonates, infants and young children. Accordingly, inhalation therapies for neonates, infants and young children focus on the use of a specialised inhalation device, such as the Babyhaler™ spacer device (Glaxo Ltd., UK) which involves the use of a face mask. However, neonates, infants and young children can be frightened by such devices and this can lead to difficulty in fitting the inhalation apparatus and maintaining it in place. Uptake of the drug through a facemask also inevitably results in the drug escaping around the sides of the mask. Accordingly, the child will not receive an optimal dose of the drug to the lungs. This results in significant and costly waste of the drug, uncertainty as to the amount of drug that has actually been administered, and can present a significant risk when the drug is being administered as a rescue therapy.

Transdermal absorption is a well recognized, although not particularly widely used, means of drug administration, which benefits from being a non-invasive and convenient way of medicating a patient. In addition, transdermal administration avoids the gastric system, meaning that it may be possible, in comparison to orally administered medication, to administer lower doses/smaller quantities of drugs via this route.

However, delivery of drugs across the skin is difficult. This is because the outer layer of the skin, the stratum corneum, forms a very effective barrier membrane that prevents most molecules from entering the body through the skin.

One strategy adopted to improve transdermal administration of topically applied compositions is the provision of a patch which is designed for attachment to the skin so that the composition is in constant contact with the skin over a period of time. For example, U.S. Pat. No. 6,211,425 discloses the use of a patch for sustained transdermal absorption of formoterol.

However, plasters and other adhesive means of topically applying drugs suffer from several disadvantages. Firstly, the adhesive used on the plasters has to be compatible with the therapeutic agent and other constituents of the composition used. This means that such plasters can be expensive and that not all therapeutic agents are suitable for inclusion in them. Secondly, the application of a plaster, particularly for long periods of time, can be both impractical and undesirable, especially for a neonate, infant or young child. A neonate, infant or child's skin is delicate and can be irritated or damaged by the application or removal of an adhesive plaster, especially if the plaster is attached to the skin for a prolonged period of time. Finally, sustained release of an active agent over a prolonged period will not impart the immediate levels of therapeutic agent necessary to provide relief in rescue situations.

In view of the drawbacks of plaster systems, application of a liquid spray or aerosol formulation to the skin for transdermal drug delivery has been proposed (the Patchless Patch® Delivery Method, Acrux DDS Pty Ltd., Australia). The formulation is said to form a non-occlusive reservoir of drug and enhancers within the skin. Dermal penetration is achieved by the incorporation of penetration enhancers into the formulations. In this regard, U.S. Pat. Nos. 6,694,777 and 6,998,138 disclose the use of at least one “safe, skin-tolerant ester sunscreen” as a penetration enhancer, with octyl salicylate disclosed as the principle agent used. In these patents, the active agents are anti-anxiety agents and anti-alopecia agents and the cumulative amount of active agent crossing the epidermis of a patient is measured in hours, with levels of approximately 2 μg/cm² and 0.05 μg/cm² achieved 5 hours after application respectively. Thus, a rapid onset of the therapeutic effect will not be observed.

The stratum corneum is composed of dead, keratin-rich cells (corneocytes) and a lipid matrix. The stratum corneum is 10-15 μm thick in adults and forms an effective barrier membrane that limits the type of molecules that can be absorbed by the skin, and also the rate of absorption. As such, transdermal administration of formulations containing therapeutic agents, has, to date, been limited.

However, in neonates, infants and young children the stratum corneum is much thinner. This means that drug absorption through the skin is increased in neonates, infants and young children, making transdermal absorption a potentially effective means of administering medication. Furthermore, the clothing of neonates, infants and young children is easily and frequently removed to expose the skin, providing opportunity for, and ease of application of a topical preparation.

It is therefore an object of the present invention to provide pharmaceutical compositions for paediatric use comprising one or more therapeutic agents, wherein the compositions can be applied topically, so as to administer the agent(s) transdermally and preferably to provide a systemic therapeutic effect.

In particular, it is envisaged that the present invention could be used in situations where a non-invasive means of drug administration is favourable, or is required. For example, where the child is too young, or in a condition where it would be inappropriate to administer medication orally or by inhalation, such as where the child is suffering from nausea and/or vomiting, or is unconscious or sub-conscious for example, when in status epilepticus, or following a general anaesthetic.

The term “neonates” as used herein refers to a child from birth to the age of about 3 months. The term “infant” as used herein refers to a child from the age of about 3 months to about 1 year. The term “young child” as used herein refers to a child between the ages of about 1 and 7 years. The term “child” as used herein refers to a human between the ages of about 7 and 14 years. The term “paediatric” as used herein refers to neonates, infants, young children and children, as defined herein.

Herein, the term “topical” means that the compositions are applied directly to the skin. Due to the nature of the compositions and the therapeutic agents, the therapeutic agents are absorbed transdermally into the bloodstream.

The term “therapeutic agent” or “pharmaceutically active agent” as used herein denotes any active substance suitable for topical application and dermal administration to a paediatric patient (particularly a human paediatric patient) in a composition or product in accordance with the present invention. Such agents include all of the drugs and classes of drugs referred to in the following passages, plus pharmaceutically acceptable equivalents thereof, such as their pharmaceutically acceptable salts, esters, prodrugs and active metabolites. Isomers of all disclosed agents are also encompassed by this disclosure.

According to a first aspect of the present invention, pharmaceutical compositions for treating a paediatric mammal are provided, the composition comprising a therapeutic agent and a pharmaceutically acceptable carrier, the composition being suitable for topical application resulting in transdermal administration of the therapeutic agent. The composition is capable of producing a systemic therapeutic effect.

Preferably, the present invention provides pharmaceutical compositions suitable for rapid paediatric transdermal administration of one or more therapeutic agents.

The term “rapid” as used herein to indicate that the absorption of a therapeutic agent into the bloodstream takes place within less than about 20, 15, 10, 5, 4, 3 or 2 minutes, or less than about 1 minute or 0.5 minutes from application to the skin. Preferably, the serum concentration of the therapeutic agent is therapeutically effective within these time periods.

In particular, this rapid speed of onset means that the compositions according to the present invention are suitable for providing rescue therapy.

The composition should be capable of being absorbed by the skin with relative ease, and absorption may be aided by rubbing the composition into the skin.

In one embodiment, the compositions of the present invention are used to treat children and young children, but not neonates or infants. In an alternative embodiment, the compositions of the present invention are used to treat infants and neonates, but not children or young children.

The compositions are for use in therapy or prophylaxis and, in particular, for use when a non-invasive means of drug administration is favourable, or is required. Conditions to be treated include, for example, epilepsy, and status epilepticus in particular, nausea and/or vomiting, gastric upset, or pain, including bone and joint pain and pain associated with inflammation or inflammatory conditions, surgical operations, migraine, cancer, etc.

The term “gastric upset” as used herein includes conditions such as irritation of the lining of the stomach, gastro-oesophageal reflux disease, acid-induced inflammation, excessive stomach acid, indigestion, gastritis, diarrhoea, and aggravation of gastric ulcers.

Therapeutic agents which may advantageously be included in the compositions include: agents which are usually administered for the treatment of status epilepticus, for example benzodiazepines such as lorazepam or diazepam, hydantoin derivatives such as phosfenytoin, or barbiturates such as pentobarbital or thiopental; agents which are usually administered for the treatment of nausea and/or vomiting, for example, selective serotonin receptor antagonists, such as ondansetron or granisetron; agents which are usually administered for the treatment of gastric upset, for example proton pump inhibitors such as omeprazole, lansoprazole, rabeprazole, pantoprazole and esomeprazole; or agents which are usually administered for the treatment of: pain and/or inflammation, for example, ibuprofen, aceclofenac, acemetacin, azapropazone, celecoxib, dexketoprofen, diclofenac, diflunisal, etodolac, etoricoxib, fenbufen, fenoprofen, flurbiprofen, indometacin, ketoprofen, mefenamic acid, meloxicam, nabumetone, naproxen, piroxicam, rofecoxib, sulindac, tenoxicam, tiaprofenic acid, valdecoxib, glyceryl trinitrate, opioids such as fentanyl, buprenorphine, salicylic acid and related salicylates.

References herein to any therapeutic agent are to be understood to include any physiologically acceptable derivative.

If desired, the compositions of the present invention may comprise more than one therapeutic agent. In such embodiments, it is important that the combined agents are compatible with one another under conditions of storage and use. One possible combination is an NSAID and an opioid, for example, diclofenac and fentanyl.

Preferably, the pharmaceutical compositions according to the present invention are provided in a convenient form. In one embodiment, the composition and its packaging allow a dose of the composition to be easily and accurately applied to the skin either by hand or using the packaging, without the need for an additional applicator. In an alternative embodiment, the composition is provided with an applicator, to allow easy and accurate administration of a dose. The packaged composition and/or the applicator are preferably small and convenient to carry around for emergency use.

The compositions are preferably provided in a package with a volume of less than about 1000 cm³, 750 cm³, 500 cm³, 250 cm³, 100 cm³, 50 cm³, 20 cm³, 10 cm³ or 5 cm³, so that the package fits easily into a Medibag™ (a first aid kit for children), handbag or pocket.

In addition, the pharmaceutical compositions are presented in such a way that will allow application of substantially all of the dose of the active agent contained therein to be administered to the patient.

“Applicator” as used herein means any device suitable for the application of a composition to the skin of a human, for example, metered dose or dose metering systems, manually operated systems, palettes, brushes, wipes, swabs or gloves.

In one embodiment of the invention, the compositions have a substantially solid form at a temperature of about 25° C. or less, and a softening point of not higher than the skin temperature of the intended subject, preferably a paediatric mammalian patient.

More particularly, the pharmaceutical composition is a substantially solid dosage form which is softenable on application thereof to an area of skin of the mammalian patient whereby, following application to the area of skin, the solid dosage form is softened to a consistency that can be substantially absorbed by the area of skin so as to effect administration of the unit dose of the therapeutic agent to the patient.

Typically, the substantially solid dosage form is provided in the form of a tablet or of a rolled or moulded preparation, for example, a pill or the like.

In one embodiment of the invention, the pharmaceutical composition is solid at a temperature of about 25° C. or less and has a softening point of not higher than 35° C., such that when the composition is placed in continuous contact with the skin of a mammalian patient, it is softened to a consistency to effect substantial application of the therapeutic agent onto a desired skin area of the mammalian patient within a time period of less than 10 minutes.

This allows for substantially complete absorption of the composition over the area of skin, so as to effect substantially complete administration of the therapeutic agent to the mammalian patient.

In such embodiments, the solid pharmaceutical composition may be provided as a unit or measured dose of a therapeutically effective amount of a therapeutic agent for treating a respiratory disorder and a pharmaceutically acceptable carrier. The unit or measured dose is preferably provided as a solid tablet, and such tablets may be packaged individually.

The term “softening point” as used herein refers to a temperature at which a substantially solid dosage form starts to soften to a consistency that can be absorbed by the skin of a patient, so as to allow transdermal absorption of the therapeutic agent present in the composition.

The softening point of a substantially solid dosage form of a pharmaceutical composition according to the first aspect of the present invention can be determined visibly as the temperature at which the substantially solid dosage form starts to soften to a consistency that can be absorbed by the skin of a patient and as such can advantageously be substantially completely absorbed by the skin of the patient so as to leave little or no undesirable residue on the skin of a patient.

Alternatively, the softening point of a substantially solid dosage form of a pharmaceutical composition according to the first aspect of the present invention can be determined using a TA-XT2 texture analyser (Stable MicroSystems Ltd., UK), suitably equipped with a 5 kg load cell. The equipment is enclosed in a temperature controlled chamber (capable of operating in the region of 60° C. to 200° C.). A tablet or other substantially solid dosage form according to the present invention may be enclosed in the chamber at the specified temperature for a time of at least 10 minutes. A 3 mm flat faced probe is pushed into the tablet or other substantially solid dosage form according to the present invention for a distance of 1 mm at a speed of 0.1 mm/sec. Measurements can be repeated at temperature increments of 1° C. and, at the temperature at which the peak force of resistance recorded (as measured by Texture Exceed software) falls to below 50% of that for a “solid” tablet or other substantially solid dosage form according to the present invention, the tablet or other dosage form is deemed to have “softened”.

The term “spreading point” as used herein refers to a temperature at which the composition has a spreading consistency. For example, the composition may flow under its own weight or at least can be spread upon the skin of a mammalian patient, for example, using finger pressure.

The mobility of a spreading composition may promote the absorption of the therapeutic agent into the skin by allowing movement of the therapeutic agent towards the skin, for example, by diffusion. The spreading point of a preparation may be measured using the TA-XT2 texture analyser mentioned above in relation to measurement of softening point and with this analyser the spreading point of a composition is the temperature at which outward flow of the composition is first observed on advance of the flat faced probe into the preparation.

In another embodiment, the pharmaceutical composition suitable for topical administration, preferably to a mammal, comprises one or more therapeutic agents and a carrier medium, wherein said preparation has a softening point of not higher than skin temperature of a mammalian patient, said composition having an aspect ratio (wall:face) of less than 1:1.

In another embodiment, the pharmaceutical composition for topical administration, preferably to a mammal, comprises a compacted granulate including one or more therapeutic agents and a pharmaceutically acceptable carrier, said compacted granulate having a softening point of not higher than skin temperature of the intended subject, preferably a paediatric mammalian patient.

In certain embodiments, the composition, which is solid prior to administration by application to the skin, has a shape to facilitate the topical application. For example, the composition can have: at least one flat surface; at least one concave surface; at least one convex surface; two flat surfaces; two concave surfaces; or two convex surfaces. The composition may be in the form of a standard tablet, spherical or half-spherical. Bullet shaped and conical shaped compositions are not preferred in the present invention.

In preferred embodiments, the compositions of the present invention have a total weight of from about 50 mg to less than 1 g, preferably from about 100 mg to about 900 mg and more preferably from about 250 mg to about 750 mg. The compositions of the present invention can have a total weight of 1 g or greater, if desired.

In compositions which are prepared for human patients, the dosage form has a softening point not higher than the normal external temperature (skin temperature) of a human. This temperature is typically not higher than about 35° C. In certain embodiments, the composition has a softening point from about 30° C. to not higher than 35° C.

In an alternative embodiment, the pharmaceutical composition contains a dose of at least one therapeutic agent for topical application to a paediatric mammalian patient, said composition being a solid during final manufacture and prior to application to an area of skin of said mammalian patient, but having a spreading consistency suitable for application to said area of skin upon exposure to (and/or contact with) the skin, said composition being individually contained in a plastic container having a removable or breakable enclosure for dispensing said unit dose.

In certain embodiments, the dosage form can be a plurality of substantially discrete substantially solid particles comprising one or more therapeutic agents admixed with a pharmaceutically acceptable carrier, said particles having a softening point of about 30° C. to about 35° C. The particles can be enclosed in a sachet, a capsule or a device suitable to dispense an individual dose of the particles.

In the embodiments discussed above where the compositions soften upon application to the skin, the carrier medium used is preferably substantially solid at a temperature of about 25° C. or less and softens to a consistency that allows for substantially complete absorption of the one or more therapeutic agents by the skin of the patient, so as to effect (preferably substantially complete) administration of the therapeutic agents to the patient, within a time period of less than about 10 minutes, preferably less than about 5 minutes, more preferably less than about 3 minutes and most preferably less than about 1 minute following application to the area of skin.

Typically, it is preferred that the carrier medium included in the substantially solid dosage form of the present invention may soften, and advantageously may be converted to a spreading consistency, at a temperature in the range of 30 to 35° C.

In its substantially solid form, the composition of the invention preferably has a size and shape suitable for application to a selected area of skin.

More particularly, it is preferred that the shape and configuration of the substantially solid dosage form is determined by the softening point of the composition and/or the carrier medium. It may be preferred that a substantially solid dosage form according to the present invention comprises a substantially unitary form; alternatively, it may comprise a plurality of discrete particles (such as a plurality of granules or the like) that can be absorbed by the skin of a mammalian patient. Preferably, the plurality of substantially discrete particles are provided in a sealed member (such as a capsule, sachet, blister package or the like) from which they are dispensed and applied to the skin of a patient.

Any component commonly used for suppositories can be used as carriers in the compositions of the present invention which soften upon application to the skin. These components include those derived from mammalian, vegetable or mineral origins, and materials partially or totally synthesized. Specific examples of such carriers include oils and fats of mammalian or vegetable origin, such as olive oil, corn oil, castor oil, cottonseed oil, wheat germ oil, cacao butter, hydrogenated oils, etc.; hydrocarbons, such as squalane, petrolatum, solid paraffin, liquid paraffin, etc.; and waxes, such as jojoba oil, carnauba wax, bees wax, lanolin, etc. Examples of partially or totally synthesized fatty acid esters include glycerol, mono-, di-, or triglycerides of medium or higher fatty acid, such as saturated linear fatty acid, for example lauric acid, myristic acid, palmitic acid, stearic acid, etc., or unsaturated linear fatty acids, for example oleic acid, linoleic acid, linolenic acid, etc. Commercially available carriers which are suitable include Witepsol (manufactured by Dynamit Nobel), Pharmasol (manufactured by Nippon Oil and Fats Co.), Isocacao (manufactured by Kao Corp.), SB (manufactured by Taiyo Oil and Fats Co.), Novata (manufactured by Henkel), Suppocire (manufactured by Gattefosse Co.), and the like. Examples of other synthetic products include polyethylene glycol, for example, macrogole, setomacrogole, etc., as well as derivatives thereof, for example, setomacrogol.

In order to obtain the desired softening point of the compositions of the present invention, different carriers can, if necessary, be combined in order to increase or decrease the softening point to obtain a suitable product. For example, in order to decrease the softening point, a plasticizer can be added, e.g., glyceryl monostearate, myristyl alcohol, polysorbate 80, propylene glycol or combinations thereof. In order to increase the softening point, a hardener can be added, e.g., beeswax, cetyl alcohol, stearic acid, stearyl alcohol, aluminium monostearate, aluminium distearate, aluminium tristearate, bentonite, magnesium stearate, colloidal silicon dioxide or combinations thereof.

A carrier for use according to the present invention may comprise any ingredient suitable for use in a pharmaceutical composition and possessing the desired properties for enabling topical administration of a dose of at least one therapeutic agent, provided that it is suitable for topical application and transdermal administration. For example, the carrier may include a cellulose or one or more ingredients selected from the group consisting of ingredients of the type suitable for use in suppositories including, for example, one or more glycerides (such as, for example, one or more glycerol esters of saturated fatty acids or one or more polyglycolysed glycerides, cocoa butter, theobroma or the like), one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like), and any of the preceding ingredients can be optionally mixed with one or more organic oils (including, for example hydrogenated vegetable oils) or the like.

It is often preferred that a carrier employed in a pharmaceutical composition according to the present invention comprises, and more preferably consists essentially of, one or more glycerides, including, in particular, one or more glycerol esters of C8-C18 fatty acids or one or more polyglycolysed glycerides.

Suitably, the carrier of a pharmaceutical composition according to the present invention comprises, or consists essentially of, a mixture of glycerides, where the glycerides can be one or more mono-, di- or tri-glycerides, optionally wherein the glycerides comprise glycerol esters of C12-C18 fatty acids. In one embodiment, the glyceride mixture is a Witepsol grade product. More particularly, the carrier may comprise, or consist essentially of, a Witepsol grade product available under any of the trade marks Witepsol H5, Witepsol H15, Witepsol H32, Witepsol S51, Witepsol S55, Witepsol S58, Witepsol W25 and Witepsol W32. In a particularly preferred embodiment, the pharmaceutical compositions according to the present invention include carriers which are Witepsol grade products available under any of the following trade marks Witepsol H5, Witepsol H15, Witepsol S51 and Witepsol S55. The Witepsol grade product available under the trade mark Witepsol H15 is particularly suitable.

In a particular embodiment of the present invention, the carrier employed in the compositions consists essentially of a Witepsol grade product substantially as described above.

Alternatively, the carrier comprises, or consists essentially of, a mixture of glycerides, where the glycerides can be selected from the group consisting of mono-, di- and tri-glycerides, the glycerides comprising glycerol esters of Cg-Cie fatty acids or one or more polyglycolysed glycerides. In one embodiment, glyceride mixtures available under the trade marks Gelucire or Suppocire are used, such as any of the following: Gelucire 33/01, Gelucire 39/01, Gelucire 43/01, Gelucire 44/14, or any of the Suppocire Standard type, Suppocire N type or Suppocire P type products.

Alternatively, the carrier used in a pharmaceutical composition according to the present invention comprises, or consists essentially of, cocoa butter.

In some embodiments, the pharmaceutical composition is spreadable, such as a cream, ointment, gel or the like. Such spreadable compositions have the advantage that they are easily applied and rubbed into the skin.

Where the composition is provided as a cream, ointment or gel, it is possible to accurately control the dose to be applied to the skin of the patient by providing one or more unit or measured doses of the spreadable composition. This helps to ensure that the patient receives an accurate, predetermined dose of therapeutic agent. In the present invention, such unit or measured doses may be packaged individually, for exampled in containers such as tubes or sachets. Alternatively, the compositions of the present invention may be dispensed by a device which is capable of dispensing an accurate, predetermined amount.

Thus, in certain embodiments the spreadable pharmaceutical composition is provided as a unit or measured dose of a therapeutically effective amount of a therapeutic agent and a pharmaceutically acceptable carrier.

Compositions of the present invention should be stored at temperatures of about 25° C. or less, in accordance with storage conditions for most pharmaceutical compositions and formulations.

In embodiments of the present invention wherein the pharmaceutical composition is provided as a cream, ointment or gel, etc., the carrier medium should allow the therapeutic agent to be carried in a stable manner. The carrier medium may have favourable organoleptic properties, for example, the composition may be water-based so as to have a non-oily feel upon application to the skin.

The carrier medium used in the spreadable compositions according to the invention should preferably allow substantially complete absorption of the therapeutic agent through the skin of the mammalian patient, so as to effect what is preferably substantially complete administration of the therapeutic agent to the patient within a time period of less than about 10 minutes, preferably less than about 5 minutes, more preferably less than about 3 minutes and most preferably less than about 1 minute or 0.5 minutes following application to an area of skin.

Any component commonly used as a base for creams, ointments or gels can be used as a carrier medium in the above described spreadable embodiments of the present invention. These components include: water; hydrocarbon oils and waxes; silicone oils; vegetable, animal or marine fats or oils; glycerides (such as, for example, or more glycerol esters of saturated fatty acids or polyglycolysed glycerides, cocoa butter, theobroma or the like) or glyceride derivatives; high molecular weight polyethylene glycol, polyoxyethylene, lanolin and derivatives thereof; fatty acids, fatty alcohols or fatty esters (including, for example, caprylic acid, caprylic triglyceride or the like); lecithin; polyhydric alcohols or esters; wax esters; sterols; phospholipids and the like. Thickening agents such as gums or other forms of hydrophilic colloids may be included. The carrier medium may comprise more than one base component.

Where the pharmaceutical composition is a cream, the carrier medium may comprise substantially more oil-based components than water. Where the pharmaceutical composition is an ointment, the carrier medium may comprise substantially more water than oil based components. Where the pharmaceutical composition is a gel, the carrier medium may substantially comprise water.

In both spreadable and solid melting embodiments, a carrier medium should be selected which is compatible with the therapeutic agent. The therapeutic agent should be chemically stable and the composition should be designed to encourage the flux of the drug from the composition through the stratum corneum.

Delivery of agents through the skin is related to Fick's law of diffusion. In this regard, the rate of flux of an agent through the skin will be governed by factors including the surface area that the composition is spread over, and the thickness of the layer applied to the skin. The rate of delivery of an agent is therefore affected by the ease with which the composition containing the agent can be spread over the surface in question.

Rapid transdermal administration is achieved by the present invention by the use of appropriate compositions which control the release of the active agent when applied to the skin, and therefore the delivery kinetics. The rate of delivery of the active agent to the skin can be modified by altering the affinity of the active agent for the carrier material compared to the affinity of the active agent for transportation through the stratum corneum. Flux from the carrier through the skin is encouraged in circumstances wherein the drug is more soluble in components of the stratum corneum and other elements found on the skin, such as sweat and sebum, than it is in the carrier. This is achieved by the preparation and use of particular carrier materials in the composition, which are tailored to the active agents in question, in order to alter the hydrophilicity of the composition. Using this approach, the solvent properties of the composition, and therefore the solubility profile of the active agent can be modified, which means that the rate of diffusion of the active agent can be controlled.

Rapid delivery of the drug through the skin may also be aided by selecting carrier materials which spread easily. In compositions according to the present invention that have a substantially solid form at a temperature of about 25° C. or less, and a softening point of not higher than the skin temperature of the intended subject, the softening time of the composition, and spreadability of the softened composition will contribute to the rate at which, and surface area of the skin over which, the composition can be spread. Rapid delivery of the drug through the skin can be achieved by use of a carrier medium which softens rapidly at temperatures not higher than skin temperature, and which spreads easily once softened.

Preferably, the carrier medium constitutes not less than about 60%, more preferably not less than about 80% and even more preferably not less than about 90%, by weight based on the weight of the pharmaceutical composition.

Pharmaceutical compositions according to the present invention may further comprise, where appropriate, additional ingredients such as one or more penetration enhancers (which may be surfactants, alcohols, esters, carboxylates, phenols, glycols or the like or any other suitable penetration enhancer), humectants, surfactants (which may be cationic, non-ionic, anionic or polymeric), emulsifiers, antioxidants, preservatives, clays, antifoaming agents, spreading agents, emollients, barriers, solubilising agents for the therapeutic agent and the like.

Pharmaceutical compositions according to the present invention may also comprise solvents, such as ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

The presence of solvents in compositions according to the present invention aids systemic administration of the therapeutic agent. The extent to which, and speed with which systemic administration of a therapeutic agent from a topically applied composition occurs is associated with the depth and rate of penetration of the therapeutic agent through the skin. The presence of solvents in compositions according to the present invention aids solubilization of the drug within the composition. Solvents for use in the present invention are also chosen in accordance with their ability to cross or bridge the stratum corneum, and in particular, the tight junctions between the corneocytes within the stratum corneum. The presence of solvents in the present invention thus enhances the rate of transdermal absorption, and the depth of penetration of the therapeutic agent, by solubilizing the agent and effecting diffusion of the agent through the stratum corneum.

Pharmaceutical compositions according to the present invention may further comprise organoleptic agents to improve the organoleptic properties of the composition. Such agents include almond oil, glycerol, linseed oil, monoethanolamine oleate, grape oil, mace oil, isopropyl myristate, isopropyl palmitate, palm kernel oil, theobroma oil, wool alcohols. The inclusion of organoleptic agents can be used, for example, to enhance the feel of the composition, which can improve patient compliance.

The use of preservatives can be undesirable, as they may provoke allergic reactions in susceptible patients, and the present invention may be advantageous in avoiding or reducing the risk of such allergic reactions. Preservatives that have been associated with allergic reactions include chlorocresol, hydroxybenzoates (parabens), polysorbates, sorbic acid and the like, and these preservatives are included in a large number of known topical compositions, including, for example, compositions available under any of the following trade marks: Drapolene, Medicaid, Siopel, Sprilon, Eurax, Efcortelan, Mildison, Fucidin H, Nystaform, Quinocort, Terra-Cortril Nystatin, Timodine, Locoid, Locoid Crelo, Modrasone, Propaderm, Betnovate, Betnovate RD, Diprosone, Dermovate, Eumovate, Trimovate, Nerisone, Haelan, Synalar, Ultralanum Plain, Zorac, Carbo-Dome, Exorex, Differin and Exelderm.

In a particularly preferred embodiment of the present invention, the pharmaceutical compositions are substantially free of the types of preservative generally included in compositions intended for dermal or transdermal administration, or at least they include such preservatives in amounts that are less than those generally required in compositions intended for dermal or transdermal administration, or they include such preservatives in amounts that generally do not provoke substantial allergic reactions in susceptible patients, substantially as hereinafter described.

The preservatives generally employed in compositions intended for dermal or transdermal administration are included to prevent or reduce contamination of such compositions. Contamination is a particular problem where a composition is repeatedly exposed to the atmosphere or is repeatedly handled. Preservatives may not be required in compositions of the present invention where the compositions are in the form of unit doses, especially if these doses are individually packaged.

Pharmaceutical compositions according to the present invention may, however, comprise one or more preservatives, such as phenoxyethanol or the like, that are included typically to substantially prevent contamination of the compositions according to the present invention during manufacture but are not generally of the type employed to prevent infection due to manual application as hereinbefore described.

In further embodiments of the present invention, the compositions are substantially free of antioxidants. In such cases, it is preferred that the compositions are packaged in a substantially inert atmosphere, such as nitrogen or the like.

The use of antioxidants can provoke allergic reactions in susceptible patients and the present invention may be advantageous in avoiding or reducing the risk of such allergic reactions in susceptible patients. Antioxidants that have been associated with allergic reactions include butylated hydroxyanisole, butylated hydroxytoluene and the like, and are known to be available in prior art topical compositions, such as those compositions available under any of the trade marks Imuderm, Siopel and the like.

In a particularly preferred embodiment of the present invention, the pharmaceutical compositions are substantially free of antioxidants of the type generally included in compositions for dermal or transdermal administration, or at least they include such antioxidants in amounts less than generally required in compositions intended for dermal or transdermal administration, or at least they include such antioxidants in amounts that generally do not provoke substantial allergic reactions in susceptible patients substantially as hereinafter described.

Antioxidants are generally employed in compositions intended for dermal or transdermal administration in order to prevent the fats present in such compositions becoming rancid and to prevent oxidation of the composition following opening of the packaging within which the composition is kept. Antioxidants may not be required in compositions of the present invention where the compositions are in the form of unit doses, especially if these doses are individually packaged.

Methods of preparing the softening compositions referred to above are disclosed in WO 02/002 03 A1, the entire disclosure of which is hereby incorporated by reference.

Tablets of the compositions may be made by normal tableting processes. For example, tablets may be compressed on a 10-station tablet press at a room/equipment temperature of 2-4° C. using either stainless steel tooling or punches suitably tipped with low adhesion materials, for example, chrome, nickel chrome, PTFE or zirconia.

According to a second aspect of the present invention, an applicator is provided for applying the compositions of the first aspect of the invention.

In one embodiment, the applicator is a pad, sponge, bar, brush, cotton ball or glove.

In another embodiment, the applicator comprises a receiving means for receiving and carrying a pharmaceutical composition and a grip for enabling a user to hold and manipulate the applicator. The grip and receiving means may be arranged such that a user holding the applicator by the grip is protected from inadvertent contact with the composition. The composition is preferably in the form of a unit or measured dose.

In further embodiments, the applicator also includes an intermediate member attached to the composition, and the receiving means of the applicator may be configured to be removably attachable to the intermediate member.

In alternative embodiments, the applicator comprises a pharmaceutical composition as substantially hereinbefore described, together with a covering member that can be arranged to substantially cover the pharmaceutical composition when the latter is applied to an area of skin of the mammalian patient, and means for adhering the covering member to an area of skin of the mammalian patient.

According to a third aspect of the present invention, a kit is provided comprising a composition of the first aspect of the present invention and an applicator. Preferably, the kit comprises at least one dose of the pharmaceutical composition.

In one embodiment, the applicator included in the kit is an applicator according to the second aspect of the present invention.

According to a fourth aspect of the present invention, a composition according to the first aspect of the present invention is provided for treating a paediatric mammalian patient wherein said composition is to be administered transdermally by topical administration of the composition to the skin of the patient.

According to a fifth aspect of the invention, there is provided the use of a therapeutically active agent in the manufacture of a medicament for transdermal administration of the therapeutic agent, wherein the therapeutic agent is for treating a mammalian paediatric patient.

The present invention will now be further illustrated by the following Examples, which do not limit the invention in any way.

### EXAMPLE 1

| Ingredients: | % w/w |
|---|---|
| Diazepam | 2 |
| Softisan 133 | 81 |
| Dry Flo AF Pure | 8 |
| Migylol 812N | 5 |
| Isopropyl Myristate | 2.5 |
| Fitoderm | 1.5 |

### EXAMPLE 2

| Ingredients: | % w/w |
|---|---|
| Omeprazole | 5 |
| Softisan 133 | 90 |
| Dry Flo AF Pure | 5 |

### EXAMPLE 3

| Ingredients: | % w/w |
|---|---|
| Ondansetron | 1 |
| Witepsol H15 | 94 |
| Migylol 812N | 5 |

Percentages are by weight based on the total weight of the combined ingredients

### Method of Preparation for Examples 1-3

In each case, all ingredients excluding the drug and dry flo were melted down until molten, and the temperature of the bulk was then maintained at 60° C. The drug and dry flo were carefully sheared into the bulk using a Silverson mixer. The bulk was then solidified by exposure to a low temperature, for example, below 15° C., preferably below 10° C. and most preferably below 4° C. The solidified bulk was then milled down and granulated, also at low temperature, for example, below 15° C., preferably below 10° C. and most preferably below 4° C.

1. A pharmaceutical composition for treating a paediatric mammalian subject, comprising a therapeutic agent, and a pharmaceutically acceptable carrier, the composition being suitable for topical application resulting in transdermal administration of the therapeutic agent to provide a systemic effect.

2. A pharmaceutical composition as claimed in claim 1, wherein said mammal is selected from one or more of a human neonate, infant, young child or child.

3. A pharmaceutical composition as claimed in claim 1, wherein the therapeutic agent is a benzodiazepine, such as diazepam.

4. A pharmaceutical composition as claimed in claim 1, wherein the therapeutic agent is a selective serotonin receptor antagonist.

5. A pharmaceutical composition as claimed in claim 1, wherein the therapeutic agent is a proton pump inhibitor.

6. A pharmaceutical composition as claimed in claim 1, further comprising one or more solvents.

7. A pharmaceutical composition as claimed in claim 6, wherein the solvent is selected from the group comprising: ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

8. A pharmaceutical composition as claimed in claim 1, further comprising one or more penetration enhancers.

9. A pharmaceutical composition as claimed in claim 8, wherein the penetration enhancer is selected from the group comprising: surfactants, alcohols, esters, carboxylates, phenols, glycols, humectants, surfactants, emulsifiers, antioxidants, preservatives, clays, antifoaming agents, spreading agents, emollients, barriers or solubilising agents.

10. A pharmaceutical composition as claimed in claim 1, wherein the composition is in the form of an ointment, cream or gel.

11. A pharmaceutical composition as claimed in claim 1, wherein the composition is a substantially solid dosage form at a temperature of about 25° C. or less and, upon contact with the skin of the subject, softens to a consistency that allows transdermal absorption of the therapeutic agent.

12. A pharmaceutical composition as claimed in claim 11, wherein the composition has a softening point of not higher than 35° C.

13. A pharmaceutical composition as claimed in claim 11, wherein the composition softens to a consistency to allow transdermal absorption of the therapeutic agent within a time period of less than 10 minutes upon being placed in continuous contact with the skin of the subject.

14. A pharmaceutical composition as claimed in claim 11, wherein the dosage form has a total weight from about 50 mg to less than 1 g from about 100 mg to about 900 mg or from about 250 mg to about 750 mg.

15. A pharmaceutical composition as claimed in claim 1, comprising a compacted granulate of the therapeutic agent and the pharmaceutically acceptable carrier, the compacted granulate having a softening point of not higher than 35° C.

16. A pharmaceutical composition as claimed in claim 1, wherein the composition comprises a plurality of substantially discrete substantially solid particles comprising the therapeutic agent admixed with a pharmaceutically acceptable carrier medium, the particles having a softening point of about 30° C. to about 35° C.

17. A pharmaceutical composition as claimed in claim 1, wherein the composition provides a therapeutically effective serum concentration of the therapeutic agent within a period of less than 10 minutes from topical application to the skin of the subject.

18. A pharmaceutical composition as claimed in claim 17, wherein the time period is less than 5 minutes, less than 2 minutes or less than 1 minute from topical application to the skin of the subject.

19. A pharmaceutical composition as claimed in claim 1, wherein the composition is free of preservatives.

20. A pharmaceutical composition as claimed in claim 1, further comprising one or more preservatives to prevent or reduce contamination of the composition during preparation.

21. A pharmaceutical composition as claimed in claim 1, wherein the composition is substantially free of antioxidants.

22. A pharmaceutical composition as claimed in claim 1, wherein the composition is for treating epilepsy, status epilepticus, nausea and vomiting, or gastric upset.

23. Use of a therapeutic agent in the manufacture of a medicament for topical application as claimed in any one of the preceding claims, the medicament being suitable for topical application resulting in transdermal administration of the therapeutic agent to provide a systemic effect.

24. An applicator for applying a pharmaceutical composition as claimed in claim 1.

25. An applicator as claimed in claim 24, comprising a receiving means for receiving and carrying the pharmaceutical composition so that the composition may be applied directly to the skin and a grip for enabling a user to hold and manipulate the applicator

26. A kit comprising at least one dose of a pharmaceutical composition for treating a paediatric mammalian subject, comprising a therapeutic agent, and a pharmaceutically acceptable carrier, the composition being suitable for topical application resulting in transdermal administration of the therapeutic agent to provide a systemic effect, and an applicator.

27. A method of treating an animal or human body comprising topically applying a pharmaceutical composition as claimed in claim 1.

28. A pharmaceutical composition or product substantially as described in any one of the Examples.

29. A pharmaceutical composition as claimed in claim 4, wherein the selective serotonin receptor antagonist is ondansetron.

30. A pharmaceutical composition as claimed in claim 5, wherein the proton pump inhibitor is omeprazole.
